(19) United States
(12) Patent Application Publication
(10) Pub. No.: US 2009/0175808 A1
(43) Pub. Date: Jul. 9, 2009

(54) COMPOSITION AND USES THEREOF

(75) Inventors: Edward Galley, Nottinghamshire (GB); David Alistair Gray, Leicestershire (GB); Stewart Paul Long, Nottinghamshire (GB); Daniel Allan White, Devon (GB); Janet Palin, Nottinghamshire (GB)

Correspondence Address: ROTHWELL, FIGG, ERNST & MANBECK, P.C., 1425 K STREET, N.W., SUITE 800, WASHINGTON, DC, 20005, US

(73) Assignee: The Boots Company PLC, Nottingham, Nottinghamshire (GB)

(21) Appl. No.: 12/297,101

(22) PCT Filed: Apr. 13, 2007
(86) PCT No.: PCT/GB07/50194

§ 371 Date: Oct. 14, 2008

Related U.S. Application Data
(60) Provisional application No. 60/898,404, filed on Jan. 31, 2007.

(30) Foreign Application Priority Data
Apr. 13, 2006 (GB) 0607478.5
Jan. 30, 2007 (GB) 0701713.0

(51) Int. Cl. A61K 8/64; A61K 38/02; A61Q 1/06; A61Q 5/00

(52) U.S. Cl. 424/59; 514/2; 424/70.1; 424/70.7; 424/64

(57) ABSTRACT

Use of a composition comprising oil bodies in the preparation of a formulation for administration to a mammal wherein said composition comprises oil curd extracted from oleaginous plant tissue, said oil curd comprising an aqueous dispersion of oil bodies and associated extrinsic protein material. Preferably, the oil curd is extracted from seed material selected from flax, sunflower, sesame, hemp, pumpkin, oilseed rape, camelina, cranberry, safflower, rosehip, blackcurrant, evening primrose, borage, meadowfoam, grapeseed and jojoba. The composition has useful properties in improving the stability of formulations, in particular in emulsifying such formulations and in improving the aesthetic acceptability of a formulation. Also, the compositions are useful in the delivery of actives into the skin &/or hair and in the regulation of the condition of the skin &/or hair. Processes for the preparation of said composition and formulations are also disclosed.

The present invention relates to the use of compositions comprising oil curd extracted from plant tissue in the preparation of formulations suitable for administration to a mammal, especially formulations for topical application; to said compositions and formulations prepared therefrom and to processes to prepare said compositions and formulations.

The skin is a complex organism and is subject to many underlying biological processes to achieve the proper functioning of the skin. The skin is also subject to the ageing process and to many extrinsic factors such as pollution, chemicals (eg on clothing), the wind, UV, external temperature extremes. Cosmetic formulations are applied to the body, especially the hair and skin, particularly the facial skin, in order to improve appearance and mitigate the effect of the intrinsic ageing process and extrinsic processes described above. Such formulations are often in the form of emulsions in order to maximise the desired effect of the ingredients utilised in the compositions. In particular, the formulations can solubilise both hydrophilic and lipophilic ingredients so that they can be transported through to the site of action. The compositions may also contain a blend of lipophilic ingredients and water-soluble humectants to moisturise the skin and maintain its barrier properties.

In order to form satisfactory emulsions, it is necessary to incorporate emulsifiers into the formulations so that an emulsion is formed which facilitates the dispersal of one phase within the other phase, is cosmetically acceptable to the user and is stable on storage. Many synthetic emulsifiers are available for such use and are widely used in modern cosmetic and toiletries. However, they have many drawbacks including potential for irritation, inactivation of other ingredients such as preservatives, impurity levels and environmental concerns. Additionally with the trend towards using naturally occurring ingredients to benefit the skin, such as plant extracts and a focus on sustainable industrial processes, it is desired to use naturally occurring emulsifiers in cosmetic formulations.

Existing naturally occurring emulsifiers may be not very effective at forming emulsions which are useful in cosmetic or dermatological formulations, are not capable of being produced by industrial processes and/or may not be stored for long periods under varying conditions of temperature and humidity. They may have poor emulsion stability where the emulsion may break into the separate phases on storage and/or when subjected to one or both of high temperature or high humidity. In addition, they may have sensory drawbacks such as poor smell and colour, be variable in their efficacy, suffer from inherent instability such as rancidity over time, only be available from limited sources or be limited in their range in terms of emulsification properties. Additionally a cosmetic formulation using such emulsifiers may not be acceptable to the user either in terms of feel, or in producing an irritant reaction, or the cosmetic emulsions may not be efficacious at delivering all the desired ingredients in the formulation to the skin.

It has been proposed that compositions comprising naturally occurring oil bodies may have valuable emulsification properties. Oil bodies act as energy stores in plant seeds for use in germination and post-germinative growth. Oil bodies produced by plants may be extracted by a variety of processes to separate them from other substances with which they are found in nature. In such processes it is important to extract the oil bodies substantially intact. Ruptured oil bodies not only reduce the overall yield but also produce an oily film which must be separated from the oil bodies.

The use of naturally occurring emulsifiers comprising oil bodies obtained from the seeds of oleaginous plants in cosmetic formulations has previously been proposed, for example a composition comprising safflower oil bodies for emulsifying cosmetic formulations, is commercially available from Lonza under the tradename Natrulon. This material comprises oil bodies which have been extracted and then subjected to a careful washing process. A typical method of preparing such washed oil bodies is described in WO-A-9853698 which describes obtaining oil bodies from a plant cell, preferably by grinding oleaginous plant seeds, removing solids from ground seeds, separating the oil body phase from the aqueous phase, washing the oil body phase to yield a washed oil body preparation; and formulating the washed oil body preparation into an emulsion. The washing step is said to remove undesirable contaminating seed proteins and the washing conditions may be selected to provide a substantially pure oil body preparation in which case only the oil body proteins are substantially present in the preparation. The process is said to yield intact oil bodies of approximately uniform size and shape. The washed oil body preparations are said to be useful in a variety of applications in which emulsions are utilised, including orally and topically applied formulations. WO-A-0030602 also describes cosmetic formulations comprising oil body preparations from oleaginous plants in combination with an active agent. The disclosure focuses on the use of washed oil bodies. The absorption or penetration of the cosmetically active agent in the oil body formulation is said to be enhanced and irritability is reduced as compared to the absence of the oil bodies. However, these oleaginous seed products involve significant washing steps, which are inconvenient and lead to extra expense.

It has also been previously proposed that oil body concentrates from oleaginous plants, particularly from the seeds/nuts of pistachio, macadamia, groundnut, hazelnut and jojoba which contain a high oil content, may be prepared without the significant washing steps required in the above disclosures and may be incorporated in suitable oil body compositions for administration to the human body. For example, the general process described in U.S. Pat. No. 5,643,583 includes crushing the seeds/nuts, emulsification in an aqueous phase, filtration and optionally centrifugation to yield an oil body concentrate. The Examples show that, in the case of macadamia oil extracted from macadamia nuts, the extracted material produced after combining ground macadamia nut material with a significant amount of water may be emulsified and then centrifuged to provide a concentrate comprising approximately 60% lipid material. The concentrate is said to have valuable emulsion properties and may be incorporated in aqueous cosmetic or pharmaceutical compositions in an amount up to 30% by weight of the composition. U.S. Pat. No. 5,683,740 relates to the same group of oleaginous plants as described in U.S. Pat. No. 5,643,583 above but produces reconstituted oil bodies by adding exogenous lipid to the ground oil cake recovered after extraction of the vegetable oils. The oil cake is said to comprise the proteins and phospholipids found in the covering of the oily bodies. The Examples show that, in the case of the oil material extracted from macadamia nuts and groundnuts, the extracted oil may be processed to provide a concentrate comprising approximately 60% lipid which may incorporated in an amount up to 30% by weight of a cosmetic or pharmaceutical composition. It has been found that the reconstituted oil bodies disclosed therein have valuable emulsification properties in cosmetic formulations.

It has now been found that a composition comprising oil curd extracted from oleaginous plant tissue, said oil curd comprising an aqueous dispersion of oil bodies and extrinsic protein material, has valuable properties in the preparation of improved formulations suitable for administration to a mammal.

Oil bodies have been extracted and characterised from a number of oleaginous sources. For example, Tzen et al, “Surface Structure and Properties of Plant Seed Oil Bodies”, J Cell Biology, 117 (1992) 327-335 describes methods to extract oil bodies from materials including maize, rape, soybean and jojoba; Tzen et al, “Lipids, Proteins and Structure of Seed Oil Bodies from Diverse Species”, Plant Physiology 101 (1993) 267-76 describes similar methods to recover oil bodies from a number of seeds, including maize, rape, mustard, cotton, flax, peanut and sesame seeds. However, there is no disclosure therein of the use of compositions comprising oil curd extracted from oleaginous plant tissue, said oil curd comprising an aqueous dispersion of oil bodies and extrinsic protein material, in improved formulations suitable for application to the human or animal body nor any suggestion that these compositions may be combined with other ingredients in an advantageous manner in a formulation to yield valuable stability, emulsifying, formulation and/or therapeutic advantages.

Accordingly, in a first aspect, the present invention provides the use of a composition comprising oil bodies in the preparation of a formulation for administration to a mammal wherein said composition comprises oil curd extracted from oleaginous plant tissue, said oil curd comprising an aqueous dispersion of oil bodies and a stability-enhancing amount of associated extrinsic protein material.

It has been found that a composition according to the invention may be an effective component in formulations for administration to a mammal, for example a human or animal body and provides advantageous properties therefor. Particular advantages have been found in the emulsification of formulation systems, especially in topical formulations, as well as improved aesthetic appearance of the formulations. Also, an improvement may be achieved in the delivery of the active ingredients through the skin and hair as well as improved regulation of the condition of the skin and hair. Furthermore, an improvement in the appearance of the skin and the hair to which formulations containing the compositions are applied may be achieved, especially in the feel of the skin and hair. The compositions can be prepared relatively easily and do not require several further purification stages.

In a preferred embodiment, there is provided the use of said composition comprising oil curd extracted from oleaginous plant tissue, said oil curd comprising an aqueous dispersion of oil bodies and a stability-enhancing amount of associated extrinsic protein material, to emulsify a formulation. Further preferably, the formulation comprises a body treatment agent (especially comprising at least one ingredient selected from a cosmetic ingredient and a pharmacologically active ingredient), and optionally a cosmetic or pharmaceutically acceptable carrier therefor. In a more preferred embodiment, the composition comprising oil curd is the sole emulsifier in the formulation.

In a further preferred embodiment, there is provided the use of said composition comprising oil curd extracted from oleaginous plant tissue, said oil curd comprising an aqueous dispersion of oil bodies and a stability-enhancing amount of associated extrinsic protein material, in a formulation (preferably a topical formulation), comprising at least one pharmacologically active ingredient to transport said active into and/or improve the delivery of the active through the skin.

In a further preferred embodiment, there is provided the use of a composition comprising oil curd extracted from oleaginous plant tissue, said oil curd comprising an aqueous dispersion of oil bodies and a stability-enhancing amount of associated extrinsic protein material in a formulation to regulate the condition of mammalian skin and/or hair. In particular, the formulation is suitable to improve the appearance of the skin and/or hair or may be used to protect the hair and/or skin, especially from environmental challenges, such as UV radiation, or external chemicals to which the skin and/or hair is exposed.

In a further preferred embodiment, there is provided the use of said composition comprising oil curd extracted from oleaginous plant tissue, said oil curd comprising an aqueous dispersion of oil bodies and a stability-enhancing amount of associated extrinsic protein material in a formulation to improve the aesthetic acceptability of said formulation.

In a further preferred embodiment, there is provided the use of said composition comprising oil curd extracted from oleaginous plant tissue, said oil curd comprising an aqueous dispersion of oil bodies and a stability-enhancing amount of associated extrinsic protein material in the preparation of a pharmaceutical formulation in the form of a tablet or a capsule.

A preferred feature of the present invention is the incorporation of said compositions comprising oil curd in formulations for administration to a mammal, for example a human or animal body, preferably topical application. Accordingly, in a second aspect, the present invention provides a formulation comprising
a) a composition comprising oil curd extracted from oleaginous plant tissue, said oil curd comprising an aqueous dispersion of oil bodies and a stability-enhancing amount of associated extrinsic protein material;
b) a body treatment agent (especially comprising at least one ingredient selected from a cosmetic ingredient and pharmacologically active ingredient); and optionally
c) a cosmetic or pharmaceutically acceptable carrier.

In a third aspect, the present invention provides an oil body-containing composition comprising oil curd extracted from oleaginous plant tissue, said oil curd comprising an aqueous dispersion of oil bodies and a stability-enhancing amount of associated extrinsic protein material. In one embodiment, said composition comprises a cosmetically and dermatologically acceptable preserving system. Said preserving system should be stable and efficacious in aqueous systems. In said embodiment, preferably the preserving system is an antimicrobial preserving system, more preferably including a preservative which does not have oxidising or reducing properties.

In an fourth aspect, the present invention provides a method of preparing a composition comprising oil curd extracted from oleaginous plant tissue, said oil curd comprising an aqueous dispersion of oil bodies and a stability-enhancing amount of associated extrinsic protein material comprising the steps of:

a) extracting oil bodies from oleaginous plant tissue material and combining with an aqueous medium;
b) removing insoluble material;
c) separating out the oil curd; and
d) storing the oil curd,
the process optionally further comprising the addition of a preserving system.

In a fifth aspect, the present invention provides a method of preparing a formulation for administration to a mammal comprising the step of incorporating a composition comprising oil curd extracted from oleaginous plant tissue, said oil curd comprising an aqueous dispersion of oil bodies and a stability-enhancing amount of associated extrinsic protein material, in said formulation. Preferably the formulation is a topical formulation, for example a cosmetic or dermatological formulation.

In a sixth aspect, the present invention provides methods for treating a mammal comprising the application of formulations comprising a composition comprising oil curd extracted from oleaginous plant tissue, said oil curd comprising an aqueous dispersion of oil bodies and a stability-enhancing amount of associated extrinsic protein material, including a method of providing an improved delivery of a pharmacologically active ingredient into the skin, a method of regulation of the condition of the skin and/or hair, a method of improving the appearance of the skin and/or hair and a method of protecting the skin and/or hair, for example from environmental or chemical challenges.

The composition comprising said oil curd, including a stability-enhancing amount of associated material comprising extrinsic protein material may be prepared by methods including milling the plant tissue, preferably the seeds forming an aqueous dispersion, filtering off any insoluble material and separating out the oil curd. The oil curd may be obtained from the oleaginous plant seed, as described in WO 9853698 above. However, the oil bodies are not subjected to the further washing steps described therein as this removes the extrinsic proteins found to be essential in accordance with the present invention and may also remove other material which may provide additional benefits to the subject compositions. The extrinsic material in combination with the oil bodies contributes to the advantageous effects of the composition of the present invention. Thus, in a useful composition comprising oil curd extracted from oleaginous plant tissue, the curd comprises an aqueous dispersion of unwashed oil bodies and associated extrinsic protein material. Unwashed oil bodies, substantially free of insoluble plant material, can be used in a formulation directly after separation of the oil curd from the extraction medium. Any insoluble plant material may be removed from the oil curd, for example, by filtration. Accordingly, the oil curd extracted from the oleaginous plant material may be used directly in a formulation once insoluble material has been removed. The oil bodies comprising lipid material may be simultaneously extracted together with the extrinsic protein material from the same oleaginous plant tissue. In the oil curd extraction process, the association between the extrinsic protein material and the oil bodies provides that the extrinsic protein material is collected together with the oil bodies and remains associated therewith during the steps of removing the insoluble material and collecting the oil curd.

The plant material from which the oil curd is derived is oleaginous plant material. Preferred classes of oleaginous plants are hemp, flax, pumpkin, sunflower, sesame, oilseed rape, camelina, cranberry, safflower, rosehip, blackcurrant, evening primrose, borage, meadowfoam, grapeseed jojoba, mustard, poppy seed, papaya, argan, maize, mango, moringa, pomegranate, passionflower, olive, hazelnut, almond, brazil nut, walnut, apricot kernel, avocado, cashew nut, pecan nut, coconut, oil palm, groundnut, pistachio, soy bean and cotton seed. More preferred classes comprise the following: hemp, flax, pumpkin, oilseed rape, sunflower, sesame, camelina, cranberry, safflower, rosehip, blackcurrant, evening primrose, borage, meadowfoam, grapeseed and jojoba. Most preferably, the oleaginous plants from which the oil curd is extracted are hemp, pumpkin, sunflower, sesame and oilseed rape.

Preferably, the oil curd is extracted from the part of the plant having the greatest concentration of oil bodies, more preferably the plant seed. Accordingly, in a preferred embodiment, the plant tissue comprises plant seed material. In one embodiment, the seeds do not include seeds in the form of nuts. The oleaginous material used in accordance with the present invention, especially the seed material, has a relatively high oil content. These oleaginous plant materials are commercially available from a number of sources.

In their naturally occurring state, oil bodies are contained in the plant tissue, especially the seeds, with other materials, including extrinsic proteins and carbohydrates. The oil bodies, also known as oleosomes, comprise a phospholipid envelope which provides an outer surface for the oil bodies, said phospholipids encasing a lipid or oily core comprising triacylglycerols. The phospholipid envelope also includes intrinsic protein material, also known as oleosins. The oleosins reside stably on the surface of the oil body and in some cases may cover substantially the whole surface of the oil body. The oleosins have a variety of functions depending on their structure, especially their hydrophilic and hydrophobic portions, which also affect their incorporation into the oil body structure. It is believed that the hydrophobic portion of the oleosin molecule penetrates the phospholipid envelope into the oily core and its amphipathic portion resides on the phospholipid layer or protrudes to the exterior. Accordingly, for the purposes of the present invention, the oleosins are considered to form an intrinsic or integral part of the oil body and provide substantially the entire intrinsic protein component of the oil body within the oil curd.

The oil curd comprises an aqueous dispersion of oil bodies in combination with protein material external to the oil body, but associated therewith. This protein material is termed herein as “extrinsic protein material”. In accordance with the present invention, the extrinsic protein material forms a part of the oil curd. It is present in the oil curd external to or outside the phospholipid/oloesin layer of the oil bodies. The extrinsic protein material has an effect on the properties of the oil bodies, but operates from outside the oil bodies. The extrinsic protein material may be loosely associated with the oil bodies through non-specific hydrogen bonds and electrostatic interactions; these forces also cause the constituents of the extrinsic material to be loosely associated together. Such association may be disrupted by means such as the addition of chaotropic agents or through changing the pH. The extrinsic protein material is generally not easily separated from the oil bodies by standard filtration procedures. Accordingly, under normal oil body extraction procedures the extrinsic protein may be carried with the oil body extract and thus recovered with the oil body extract. If it is desired to separate the extrinsic protein from the oil bodies, ingredients may be required which break the bonds between the oil bodies and the extrinsic material, for example electrolytes, such as sodium chloride, or chaotropic materials, such as urea. Typical extrinsic proteins vary between plant varieties and are known to the person skilled in the art, but include albumins, globulins, prolamines and glutelins.

The oil curd composition is generally in liquid form as the oil curd comprises an aqueous dispersion of oil bodies, however, the composition may be subsequently dried, for example by freeze drying. The liquid dispersion of oil bodies in an aqueous phase may also be considered an emulsion, for example an oil-in-water emulsion. The composition may comprise other components, for example, liquid carrier components as described hereinafter. The oil curd generally forms from 10% to 100% by weight of the composition. In preferred compositions according to the present invention, the oil curd is present to an extent of at least 90% by weight of the composition, preferably at least 95% by weight, most preferably at least 99% by weight of the composition. In one embodiment, the oil curd may form substantially the entire composition, ie the composition may consist essentially of oil curd. The total concentration of oil curd in the composition according to the present invention may therefore fall in the range from 90% to 100% by weight, more preferably from 90 to 99% by weight, most preferably from 95 to 99% by weight.

Preferably, the oil bodies in the oil curd comprise lipid material in an amount from 90% to 99% by weight of the oil bodies, more preferably from 94% to 98% by weight lipid. The lipid is mainly composed of triacylglycerols particular to the plant material used. Preferably the lipid comprises from 90% to 99.9% by weight triacylglycerols, more preferably from 94% to 99% by weight and most preferably from 96% to 98% by weight triacylglycerols.

Preferably, the oil curd composition comprises lipid material in an amount greater than 5% by weight of the oil curd, more preferably greater than 15% by weight and most preferably greater than 25% by weight. In one embodiment, the oil curd composition comprises less than 70% by weight or less than 65% by weight lipid. Preferably the oil curd composition comprises the lipid in an amount less than 60% by weight of the oil curd, more preferably less than 55% by weight and most preferably less than 50% by weight. Accordingly, the lipid is preferably present in an amount of from 5% to 65% by weight of the oil curd, more preferably 15% to 55% by weight and most preferably 25% to 50% by weight.

The amount of lipid or oil from the oil bodies may also be defined in terms of the dry weight of the oil curd composition wherein all the water from the curd has been removed. The dry oil curd preferably comprises lipid in an amount from 45% to 90% by weight of the dry oil curd, preferably from 55% to 80% by weight and most preferably from 65% to 80% by weight.

The phospholipid is preferably present in an amount of from 0.1% to 5% by weight of the oil bodies, more preferably 0.3% to 4% by weight and most preferably 0.6% to 2% by weight. Preferably, the phospholipid is present in an amount of from 0.05% to 5% by weight of the oil curd composition, more preferably from 0.1% to 3% by weight and most preferably from 0.3% to 1% by weight.

Preferably, the intrinsic protein material or oleosins form greater than 0.1% by weight of the oil bodies, more preferably greater than 0.3% by weight and most preferably greater than 0.6% by weight of the oil bodies. Preferably, the intrinsic protein material forms less than 5% by weight of the oil bodies, more preferably less than 4% by weight and most preferably less than 2% by weight of the oil bodies. Accordingly, the intrinsic protein material is preferably present in an amount of from 0.1% to 5% by weight of the oil bodies, more preferably from 0.3% to 4% by weight and most preferably from 0.6% to 3% by weight.

The intrinsic protein material is preferably present in an amount of from 0.05% to 5% by weight of the oil curd composition or aqueous dispersion, more preferably from 0.1% to 3% by weight and most preferably from 0.2% to 2% by weight.

The amount of intrinsic protein material may also be defined in terms of the dry weight of the oil curd wherein all the water from the composition has been removed. The dry oil curd preferably comprises intrinsic protein in an amount from 0.02% to 6% by weight of the dry oil curd, preferably from 0.1% to 5% by weight and most preferably from 0.6% to 3% by weight.

The oil curd aqueous dispersion suitably comprises the extrinsic protein in a stability-enhancing amount. This amount of extrinsic protein material provides a formulation with enhanced stability properties over prior oleaginous oil body compositions, such as disclosed in U.S. Pat. No. 5,643,583. It has been found that the extrinsic protein provides advantageous properties in providing a stable emulsion which may lead to the various benefits of the invention. The oil curd aqueous dispersion preferably comprises the extrinsic protein in an amount greater than 1% by weight of the oil curd, more preferably greater than 2% by weight, especially greater than 5% by weight and most preferably greater than 10% by weight. The oil curd preferably comprises extrinsic protein in an amount less than 35% by weight of the oil curd, more preferably less than 30% by weight, especially less than 25% by weight and most preferably less than 20% by weight. Thus, a preferred composition comprises oil curd comprising from 1% to 35% by weight extrinsic protein, more preferably from 2% to 30% by weight, especially from 5% to 25% by weight and most preferably from 10% to 20% by weight.

The amount of extrinsic protein may also be defined in terms of the dry weight of the oil curd wherein all water has been removed from the composition. The dry oil curd preferably comprises extrinsic protein in an amount from 1% to 40% by weight extrinsic protein, preferably from 2% to 30% by weight and most preferably from 3% to 25% by weight.

As hereinabove described, the total protein content of the oil curd is provided by protein material intrinsic to the oil bodies and protein material extrinsic to the oil bodies. Preferably, the amount of extrinsic protein material exceeds the amount of intrinsic protein material; thus the extrinsic protein forms the major component of the total protein content of the oil curd. Suitably, the oil curd aqueous dispersion comprises a total protein content of from 1% to 35% by weight of the oil curd, preferably from 2% to 30% by weight, more preferably from 3% to 20% by weight and most preferably from 4% to 15% by weight. The total protein content of the oil curd will be partly determined by the amount of the aqueous phase present; accordingly the total protein content of the oil curd may also be determined in terms of its dry weight wherein all the water from the composition has been removed. Preferably the total protein content is from 1% to 40% by weight dry oil curd, more preferably from 2% to 30% and most preferably from 3% to 30% by weight. Preferably, the total protein content of the curd is made up from 2% to 40% intrinsic proteins and from 60% to 98% extrinsic proteins, more preferably from 3% to 25% intrinsic proteins and from 75% to 97% extrinsic proteins, most preferably from 5% to 20% intrinsic proteins and from 80% to 95% extrinsic proteins. The ratio of intrinsic protein to extrinsic protein is preferably in the range 1:1 to 1:50 parts by weight, more preferably in the range 1:1 to 1:30 parts by weight and most preferably 1:1 to 1:10 parts by weight.

The ratio of the lipid or oil from the oil bodies to the total protein content of the composition is preferably less than 50:1 parts by weight, more preferably less than 30:1, especially less than 15:1 and most preferably less than 10:1 parts by weight. The ratio of the lipid to the total protein content of the composition is preferably greater than 1:10 parts by weight, more preferably greater than 1:2 and most preferably greater than 1:1 parts by weight. Advantageous compositions contain a ratio of the lipid to the total protein in the range 30:1 to 1:2 parts by weight, more preferably in the range 20:1 to 1:1 parts by weight and most preferably 10:1 to 1:1 parts by weight.

Advantageous compositions contain a ratio of the lipid to extrinsic protein in the range 40:1 to 1:10 parts by weight, more preferably in the range 30:1 to 1:2 parts by weight, particularly 20:1 to 1:1 parts by weight and most preferably 10:1 to 1:1 parts by weight. In especially preferred compositions, the lipid material present in the oil curd exceeds the amount of extrinsic protein material present in the oil curd.

Preferably, the oil bodies used in accordance with the present invention are recovered intact from the oleaginous plant tissue; accordingly the majority are present in the composition as substantially spherical, intact oil bodies. Preferably greater than 85% by weight or more, more preferably greater than 95% oil bodies, are present in the composition in intact form. The oil bodies have differing sizes according to the plant material from which they are derived. Preferably, the mass median diameter (d50) is in the range from 0.1 to 10 μm, preferably 0.5 to 6 μm.

A composition according to the invention comprises an aqueous dispersion of oil bodies, material extrinsic to the oil bodies and optionally further material dispersed or solubilised in the aqueous phase. In the process to extract the oil curd from the oleaginous plant tissue, the aqueous phase, for example water, may be added to the plant tissue before and/or during the extraction process. The amount of aqueous phase added may be chosen within known parameters to facilitate the extraction process and will depend on the process parameters, including the equipment used, the scale and speed of the extraction process and the quantity of materials. The amount of water in the composition may vary according to the extraction process and the intended use of the composition. Accordingly, at least a portion of the aqueous phase is added during the extraction of the oil curd from the oleaginous plant tissue. Conveniently, substantially the whole of the aqueous phase is provided from the extraction process, in which case the composition consists essentially of oil curd. However, a portion of the aqueous phase may be added after the extraction process according to a desired ratio of aqueous phase to oil bodies. Thus the composition may comprise at least a portion of exogenous water.

The ratio of oil bodies to aqueous phase is preferably in the range 10:1 to 1:10 parts by weight, more preferably in the range 5:1 to 1:5 parts by weight and most preferably 2:1 to 1:2 parts by weight. The aqueous phase preferably forms greater than 10% by weight of a composition according to the present invention, more preferably greater than 20% by weight, most preferably greater than 25% by weight and especially greater than 30% by weight. The aqueous phase preferably forms less than 90% by weight of the composition, more preferably less than 80% by weight, most preferably less than 70% by weight and especially less than 50% by weight. Thus, a preferred composition comprises from 10% to 90% aqueous phase by weight of the composition, more preferably from 12% to 80% by weight, most preferably from 18% to 60% by weight and especially from 15% to 55% by weight. Conveniently, water provides the continuous phase in the composition. Preferably, the aqueous phase comprises water in an amount from 50% to 90% by weight of the aqueous phase, more preferably from 60% to 85% by weight and most preferably from 70% to 80% by weight. Accordingly, preferably the water forms at least 10% by weight of the oil curd composition, more preferably at least 12% by weight and most preferably at least 15% by weight and especially at least 20% by weight. In further preferred compositions, the amount of water present is less than 90% by weight of the composition, more preferably less than 80% by weight, most preferably less than 70% by weight and especially less than 60% by weight. The total concentration of water in a composition according to the present invention may therefore fall in the range from 10% to 90% by weight of the composition, more preferably from 12% to 80% by weight, most preferably from 18% to 60% by weight and especially from 15% to 55% by weight.

A preferred embodiment of the invention comprises oil curd extracted with water as the extraction medium. The amount of carbohydrate in the oil curd extracted in the absence of a carbohydrate-containing extraction medium will not generally be significant, for example less than 1% by weight of the oil curd. However, in another embodiment, the oil curd may be extracted with an extraction medium comprising a carbohydrate material. In this case, the oil curd prepared in accordance with this invention may also comprise carbohydrate material, preferably in the aqueous phase. A proportion of the oleaginous plant tissue may be made up of saccharides. Accordingly, at least a portion of the carbohydrate may be provided from the plant tissue.

However, the carbohydrate may also comprise at least a portion of exogenous carbohydrate provided during the extraction process. Said exogenous carbohydrate may form the major proportion of the carbohydrate material, for example from greater than 50% by weight of the total carbohydrate material to 99% by weight, especially, greater than 80% to 98% by weight. The carbohydrates may comprise one or more of the following materials: monosaccharides such as glucose or fructose, disaccharides such as sucrose and polysaccharides such as starch and cellulose. Examples of complex polysaccharides also include non-cellulose derivatives containing the following monomer units: xyloglucans, xylans, arabinogalactans, galactose, arabinose, uronic acid and xylose.

If present, preferably, the carbohydrate content of the oil curd according to the present invention comprises from 0.01% to 30% by weight of the oil curd aqueous dispersion, more preferably from 0.05% to 20% by weight, most preferably from 0.1% to 10% by weight of the oil curd and especially from 0.5% to 5% by weight of the oil curd. Carbohydrate external to the oil bodies preferably provides substantially the total carbohydrate content of the oil curd.

The amount of carbohydrate material may also be defined in terms of the dry weight of the oil curd wherein all the water has been removed from the composition. If present, the dry oil curd preferably comprises carbohydrate in an amount from 0.1% to 40% by weight extrinsic protein, preferably from 0.5% to 30% by weight and most preferably from 0.5% to 25% by weight.

If present, preferably, the ratio of carbohydrate material to total protein content in the oil curd is in the range 10:1 to 1:10 parts by weight, more preferably in the range 5:1 to 1:5 parts by weight and most preferably 2:1 to 1:2 parts by weight.

Further materials such as a preserving system may be incorporated in a composition according to the present invention. Such ingredients may include preserving systems, including preservatives, such as 2-bromo-2-nitropropane-1,3-diol (bronopol, which is available commercially under the trade name Myacide®), benzyl alcohol, diazolidinyl urea, imidazolidinyl urea, methyl paraben, ethyl paraben, phenoxy ethanol, propyl paraben, sodium methyl paraben, sodium ethyl paraben, sodium dehydroacetate, polyhexamethylenebiguanide hydrochloride, isothiazolinone, Phenonip® and sodium propyl paraben, preferably in an amount from 0.001% to 5% by weight, more preferably 0.01% to 1% by weight of the composition. In a preferred embodiment, the composition comprises said oil curd aqueous dispersion and a preserving system, more preferably the composition consists essentially of said oil curd aqueous dispersion and a preserving system.

The compositions of the present invention may also include further conventional formulating components, including but not limited to, viscosity and pH modifying, stability-enhancing, perfuming and colouring agents and other ingredients as desired and as described hereinafter, typically, but not limited to, an amount up to 10% by weight of the composition, for example 0.0001% to 10% by weight of the composition. Each ingredient is used in a conventional amount in relation to its intended use in the composition. Such ingredients may also include further processing aids preferably in an amount from 0.001% to 5% by weight, more preferably 0.01% to 1% by weight of the composition.

The formulations comprising the compositions according to the present invention are preferably presented in the form of a cream, gel cream, lotion, waxy solid, serum, ointment, salves, pastes, sprays and milks.

The composition comprising oil curd is incorporated into a formulation in an amount to achieve the desired effect. Preferably the oil curd composition is present in an amount from 0.01% to 40% by weight of the formulation, more preferably 0.1% to 20% and most preferably 5% to 15% by weight.

Preferably, the formulation in which said composition comprising oil curd is combined is a topical formulation for application to the skin or the hair and may contain a body treatment agent (especially comprising at least one ingredient selected from a pharmacologically active ingredient and a cosmetic ingredient) adapted to regulate the condition of the skin and hair. Preferably, the formulation is a skincare composition, a haircare composition or a colour cosmetic composition. Suitably, the skin care formulation is a face cream or lotion, an eye cream, a hand cream or lotion, a body cream or lotion, a foot cream or lotion, a lip balm, a cleanser, a moisturiser, a face mask, a serum, a body cleansing cream, a face/body/foot scrub, a balm, a sunscreen cream or lotion, an after-sun cream or lotion, a fake tan cream or lotion and a nappy cream or lotion. The hair care formulation may be a conditioner, a balm, a wax, a putty, an emulsion spray, a cleansing product or a serum. The colour cosmetic formulation may be a lipstick, a lip gloss, a foundation, a mascara, an eye cream including eyeshadow or a blusher.

Suitable skin care active ingredients include anti-acne actives eg salicylic acid, vitamins eg Vitamin A or retinol, anti-wrinkle or anti-ageing actives such as peptides, flavonoids and promoters of collagen and/or elastin production, hydroxy acids, anti-oxidants, anti-inflammatories/soothing agents, anti-microbials eg triclosan, skin-lightening actives such as mulberry or ascorbic acid derivatives, anti-fungals, skin conditioners eg panthenol, organic or inorganic sunscreens eg butyl methoxydibenzoylmethane and titanium dioxide, plant extracts and the active principals from these, skin identical lipids such as ceramides, moisturisers and materials to promote the repair of the skin barrier such as hyaluronic acid, anti-cellulite actives, artificial tanning agents, promoters of melanin synthesis, skin tensors, myorelaxants, skin nutrients such as amino acids, sebum regulators, anti-redness actives, regulators of microbial growth and other suitable materials that are known to those skilled in the art.

Suitable hair care active ingredients include silicones, vitamins, hair conditioners, shine agents, resins, temporary, semi-permanent and oxidative dyes, sunscreens, antioxidants, proteins, amino acids, organic acids, anti-dandruff agents, lipids, moisturisers, humectants, film formers, reducing agents, oxidising agents and other suitable materials that are known to those skilled in the art.

The formulation may also comprise pharmaceutical actives including analgesics/anti-pyretics, anti-asthmatics, antibiotics, anti-depressants, anti-diabetics, anti-fungal agents, anti-hypertensive agents, anti-inflammatories, anti-neoplastics, anti-anxiety agents, immunosuppresants, anti-migraine agents, sedatives/hypnotics, anti-angina agents, anti-arrythmics, anti-arthritis agents, anti-coagulants, thrombolytic agents, anti-convulsants, anti-Parkinson agents, anti-histamines/anti-pruritics, anti-bacterial agents, anti-viral agents, bronchodilators, steroids and hormones, hypoglycaemic agents, hypolipidemic agents, proteins, anti-ulcer/anti-reflux agents, anti-emetics/anti-nausea agents, local anaesthetics, decongestants, tussives, expectorants and insect repellents.

The conventional amounts of these active ingredients, used individually or in combination with other active ingredients, will be known to those skilled in the art. Therapeutically effective quantities to treat the desired symptom or symptoms will be used in the formulations according to the present invention. Typically, each active ingredient may be used in the formulation in the range from 1×10⁻⁶ to 80% by weight of the formulation, but more usually will be in the range from 0.001% to 20% by weight, preferably from 0.01% to 8% by weight and most preferably 0.01% to 5% by weight of the formulation.

The oil curd may be used to emulsify a lipophilic ingredient in the oil curd composition to provide a stable formulation, especially for a body treatment agent. In addition, certain lipophilic ingredients, as indicated herein, are useful as skin and/or haircare agents, for example as emollients or skin barrier treatments.

Preferably, the formulation comprises a carrier, which may comprise a hydrophilic component and/or a lipophilic ingredient. More preferably, the carrier comprises at least one hydrophilic component and at least one lipophilic component. Most preferably the carrier is suitable for topical application.

Preferably, the hydrophilic component comprises water. Further preferably, the formulation comprises an aqueous emulsion, for example an oil-in-water emulsion, preferably comprising 60% to 95% by weight water, more preferably 70% to 90% by weight water, most preferably 75% to 85% by weight water.

Typically, the lipophilic material comprises one or more materials selected from the following classes: natural oils, hydrocarbons, emollient esters, silicones, fatty alcohols and waxes. Examples of these are as follows: Natural oils such as almond, sunflower, wheatgerm, olive, jojoba, rosehip and apricot kernel; Non-volatile hydrocarbons such as mineral oils, petrolatum, squalane, squalene and hydrogenated polyisobutene; Emollient esters such as isopropyl mysristate, isopropyl palmitate, isononyl isononanoate and C₁₂₋₁₅ alkyl benzoate; Silicones such as cyclic or linear volatile silicone eg cyclopentasiloxane, linear dimethicones, silicone gums, silicone resins and silicone elastomers; Volatile hydrocarbons such as isododecane, isohexadecane and isoparaffins; Fatty alcohols such as cetyl alcohol and stearyl alcohol; and Waxes such as candellia wax and microcrystalline wax.

Suitable oils for the oil phase of the oily dispersions and the oil phase of the oil-in-water emulsions or multiple emulsions (for example oil-in-water-in-oil) of the present invention may comprise for example:

a) hydrocarbon oils such as paraffin or mineral oils;
b) waxes such as beeswax or paraffin wax;
c) natural oils such as sunflower oil, apricot kernel oil, shea butter or jojoba oil;
d) silicone oils such as dimethicone, cyclomethicone or cetyidimethicone;
e) fatty acid esters such as isopropyl palmitate or isopropyl myristate;
f) fatty alcohols such as cetyl alcohol or stearyl alcohol; or
g) mixtures thereof, for example, the blend of waxes available commercially under the trade name Cutina (Henkel).

Typically, if used, the oil content may comprise up to 30% by weight of the formulation, for example from 0.1% to 30% by weight, preferably 1% to 15% by weight.

Although it is a particular advantage of the present invention that the formulations do not need an additional emulsifier, optionally one or more additional emulsifiers known in the art for use in oil-in-water emulsions may be employed. It has been found that particularly effective oil-in-water formulations can be prepared by using an emulsifier or mixture of emulsifiers selected from known cosmetically acceptable emulsifiers which include:

a) sesquioleates such as sorbitan sesquioleate, available commercially for example under the trade name Arlacel 83 (ICI), or polyglyceryl-2-sesquioleate;
b) ethoxylated esters of derivatives of natural oils such as the polyethoxylated ester of hydrogenated castor oil available commercially for example under the trade name Arlacel 989 (ICI);
c) silicone emulsifiers such as silicone polyols available commercially for example under the trade name ABIL WS08 (Th. Goldschmidt AG);
d) anionic emulsifiers such as fatty acid soaps e.g. potassium stearate and fatty acid sulphates e.g. sodium cetostearyl sulphate available commercially under the trade name Dehydag (Henkel);
e) ethoxylated fatty alcohols, for example the emulsifiers available commercially under the trade name Brij (ICI);
f) sorbitan esters, for example the emulsifiers available commercially under the trade name Span (ICI);
g) ethoxylated sorbitan esters, for example the emulsifiers available commercially under the trade name Tween (ICI);
h) ethoxylated fatty acid esters such as ethoxylated stearates, for example the emulsifiers available commercially under the trade name Myrj (ICI);
i) ethoxylated mono-, di-, and tri-glycerides, for example the emulsifiers available commercially under the trade name Labrafil (Alfa Chem.);
j) non-ionic self-emulsifying waxes, for example the wax available commercially under the trade name Polawax (Croda);
k) ethoxylated fatty acids, for example, the emulsifiers available commercially under the trade name Tefose (Alfa Chem.); or
l) mixtures thereof.

It is preferred that the composition is free of surfactants that do not have an emulsifying action in formulations according to the present invention as these may affect the structure of the oil bodies. Accordingly, in a preferred embodiment, the invention provides a composition substantially free of external surfactants, ie less than 2% w/w, preferably less than 1% w/w, most preferably less than 0.1% w/w of the composition.

If desired, the oil curd composition may be combined with a cationic material, preferably a cationic conditioning agent.

Thickeners, viscosity modifying agents and/or gelling agents may be added to the subject formulations, such as acrylic acid polymers e.g. available commercially under the trade name Carbopol (B.F. Goodrich) or modified celluloses e.g. hydroxyethylcellulose available commercially under the trade name Natrosol (Hercules) or hydroxypropylmethyl cellulose, amine oxides, block polymers of ethylene oxide and propylene oxide (for example, those available from BASF Wyandotte under the trade name “Pluronic”®), PVM, MA, or a decadiene crosspolymer (available under the trade name Stabilez 60), ethoxylated fatty alcohols, salt (NaCl), phthalic acid amide, polyvinyl alcohols, fatty alcohols and alkyl galactomannans available under the trade name N-Hance from Hercules, suitably in an amount of from about 0.5% to about 10% by weight of the composition.

Sequestering agents may be added to the formulation, such as ethylenediamine tetraacetic acid and salts thereof, suitably in an amount of from about 0.005% to about 0.5% by weight of the composition.

The formulation may also include waxes such as cocoa butter, suitably in an amount of from about 1% to about 99% by weight of the composition.

The formulation may also comprise suitable cosmetically acceptable diluents, carriers and/or propellants such as dimethyl ether.

The formulation may also include pearlising agents such as glycol stearate, suitably in an amount of from about 0.01% to about 10% by weight of the composition.

Perfumes may be added suitably in an amount of from about 0.01% to about 2% by weight of the formulation, as may water soluble dyes such as tartrazine, suitably in an amount of from about a trace amount (such as 1×10⁻⁵%) to about 0.1% by weight of the formulation.

The formulation may also include pH adjusting agents such as sodium hydroxide, aminomethyl propanol, triethanolamine, suitably in an amount of from about 0.01% to about 10% by weight of the formulation.

The formulation may be buffered by means well known in the art, for example by use of buffer systems comprising succinic acid, citric acid, lactic acid, and acceptable salts thereof, phosphoric acid, mono- or disodium phosphate and sodium carbonate. Suitably, the formulation may have a pH between about 3 and about 10, preferably between about 4 and about 8. The formulations of the present invention may additionally comprise other cosmetically useful ingredients which will be well known to those skilled in the art. These include, for example, humectants such as glycerin or 1,3-butylene glycol; antioxidants such as DL-α-tocopheryl acetate or butylated hydroxytoluene; emulsion stabilising salts such as sodium chloride, sodium citrate or magnesium sulphate; film formers to assist spreading on the surface of the skin such as alkylated polyvinylpyrrolidone e.g. available commercially under the trade name Antaron (GAF); powders such as silica, PMMA, mica, polyethylene; polymer powders such as polyurethanes and polyamides, boron nitride, Teflon, serecite; colourings and pigments such as titanium dioxide, iron oxides and lakes; dyes such as water soluble dyes including FD & C Blue no 1; exfoliating materials such as polyethylene beads, loofah, crushed apricot seeds, crushed walnut seeds, bamboo powder, aluminium oxide, silica, oatmeal, rice flour, crushed olive stones and poppy seeds; flavours, fragrances and essential oils. The formulation ingredients described herein may be present in conventional amounts well known to those skilled in the art according to the intended use of the ingredient in the formulation. For example, typically, each ingredient is used in (but is not limited to) an amount up to 10% by weight of the composition, for example 0.0001% to 10% by weight of the formulation.

Preservatives may be added to the formulations according to the invention, such as 2-bromo-2-nitropropane-1,3-diol (bronopol, which is available commercially under the trade name Myacide®), benzyl alcohol, diazolidinyl urea, imidazolidinyl urea, methyl paraben, ethyl paraben, phenoxy ethanol, propyl paraben, sodium methyl paraben, sodium ethyl paraben, sodium dehydroacetate, polyhexamethylenebiguanide hydrochloride, isothiazolinone and sodium propyl paraben, suitably in an amount of from about 0.01% to about 10% by weight of the composition.

In a process to prepare the oil curd composition, a) the oil bodies and associated extrinsic protein material are extracted from the oleaginous plant tissue, preferably by a milling process, b) insoluble plant tissue removed, c) the oil curd collected and d) stored. A preserving system may be added at a convenient point in the above process.

Optionally, the seeds may be soaked in extraction media before or after stage (a).

In step (a), the plant tissue, preferably seeds, may be wet or dry milled in a process which produces fine particles of plant tissue and enables release of the oil bodies from the plant tissue, but minimises the breakage of the oil bodies. Preferably, the method comprises wet-milling the plant tissue, especially the plant seeds. A preservative may be added at this stage. In step (b), the unwanted plant tissue material may be separated from the oil curd using aqueous extraction media. The plant tissue, especially the seeds, may be combined with the extraction media before, during or after milling, preferably before milling. The unwanted fibrous material is separated from the plant tissue by filtration or other known separation means. In step (c), the oil curd is preferably separated from the extraction media by centrifuging. Suitably, the oil curd is separated off from the extraction media either by skimming the curd from the surface of the extraction media or removing the extraction media from underneath the curd. A preservative system may be added to the oil curd at this stage (Step (d)). After extraction, the oil curd is preferably stored at 0-25° C.

In a preferred process, in step (a), the oleaginous seeds are soaked in extraction media and wet milled. This may be achieved by known techniques. The combination of plant tissue and extraction media optionally comprises a preserving system. The extraction media typically comprises an aqueous dispersion for example water, optionally in combination with a buffer, such as a phosphate salt, for example sodium phosphate. Conveniently, the pH of the mixture may be maintained between pH 6 and 9. The ground material is homogenised in extraction media by a blender, eg a Kenwood blender, for an appropriate period to release the intact oil bodies from the insoluble material and to emulsify the oil bodies. In filtration step (b), the mixture comprising oil curd, insoluble plant tissue and extraction media is filtered through a suitable filter material, for example through cheesecloth or other suitable material, or other separation means, which allows the passage of the oil curd aqueous dispersion, but not the insoluble plant tissue material, therethrough and the filtrate is collected. In step (c), the oil curd is substantially separated from the extraction media. Suitably, the extraction media has a different density than the oil curd and the oil curd can be separated therefrom by known techniques, such as centrifuging. The purpose of centrifugation is to substantially separate the oil curd phase from the aqueous extraction media phase. The oil curd comprising the oil bodies and extrinsic material preferably floats to the top of the homogenate and is carefully removed from the centrifugation liquid or extraction media to yield the oil curd. In the curd, the oil body phase may contain a proportion of the aqueous extraction media through interaction between the components, for example the aqueous extraction media may be held between the oil bodies. In step (d), a preserving system as previously described may then be added to the oil curd and the composition stored at 0-25° C. Optionally, further exogenous aqueous phase, including water, may be added.

There may also be provided a composition comprising oil curd extracted from oleaginous plant tissue, said oil curd comprising an aqueous dispersion of oil bodies and an extrinsic protein material and/or carbohydrate material. Comments made herein may apply to compositions containing carbohydrate material with or without extrinsic protein material.

The composition comprising oil curd according to the present invention may also be used for additional non-topical purposes known to those skilled in the art, eg in food products, non-topical healthcare pharmaceutical formulations, including toothpastes.

The invention will now be described in greater detail, by way of illustration only, with reference to the following Examples.

### Preparation of Oil Curd Used in the Examples

The oil curd was extracted from the following seeds: sunflower, sesame, hemp, pumpkin, rape according to the method described below.

Intact oil seeds were added to extraction media (1:5, w/v; 10 mM sodium phosphate buffer pH 7.5, 1% phenonip v/v) then homogenised (Kenwood BL315 blender; 3 min). The slurry was filtered through three layers of cheesecloth and the filtrate centrifuged at 9,000 RPM/14,300×g (Beckman J2-21 centrifuge; fixed rotor JA-10) for 30 min at 10° C. The oil curd which floated to form a creamy pad on the top of the homogenate, was carefully removed and stored at 5° C.

It was found that the curds (pH 7.5) had the following moisture, lipid and protein contents as shown in Table 1 below:

TABLE 1

| | % Moisture Content | % Lipid Content | % Protein Content |
|---|---|---|---|
| Sunflower | 45 ± 2 | 37 ± 2 | 14 ± 1 |
| Sesame | 37 ± 1 | 49 ± 4 | 5 ± 1 |
| Hemp | 32 ± 1 | 48 ± 2 | 5 ± 1 |
| Pumpkin | 18 ± 1 | 65 ± 2 | 3 ± 1 |
| Oil Seed | 49 | 36 | 8 |
| Rape | | | |

The ratio Lipid to Protein was calculated to be as follows:

| | |
|---|---|
| Sunflower | 2.64:1 parts by weight |
| Sesame | 9.8:1 parts by weight |
| Hemp | 9.8:1 parts by weight |
| Pumpkin | 21.6:1 parts by weight |
| Oilseed rape | 4.5:1 parts by weight |

The amount of extrinsic protein material may be calculated by subtracting the amount of intrinsic protein (oleosins) from the total protein content.

It was also found that the particle sizes of the oil bodies (Mean Mass median diameter) were as follows:

| | |
|---|---|
| Sunflower | 3.4 μm |
| Sesame | 5.4 μm. |
| Hemp | 3.2 μm. |
| Pumpkin | 2.6 μm. |

The curd was dried by a conventional freeze drying process. The lipid and protein content of the dry curd composition is shown in Table 2 below.

TABLE 2

| | % w/w Lipid Content | % w/w Protein Content |
|---|---|---|
| Sunflower | 67 ± 3 | 26 ± 1 |
| Sesame | 77 ± 6 | 7 ± 1 |
| Hemp | 71 ± 2 | 8 ± 2 |
| Pumpkin | 79 ± 2 | 3 ± 1 |

On drying the oil curd, the relative ratio between the lipid and protein was found to be as follows:

| | Ratio Lipid to Protein | |
|---|---|---|
| Sunflower | 2.58:1 | parts by weight |
| Sesame | 11:1 | parts by weight |
| Hemp | 8.88:1 | parts by weight |
| Pumpkin | 26.34 | parts by weight |

### EXAMPLE 1

### Sunscreen

| | % w/w |
|---|---|
| Oil curd | 8.00 |
| C12-15 alkyl benzoate | 5.00 |
| Butylmethoxy dibenzoyl methane | 2.90 |
| Ethylhexyl methoxycinnamate | 1.90 |
| Octocrylene | 8.00 |
| Methylene bis-benzotriazole tetramethylbutylphenol | 0.20 |
| Almond oil | 1.00 |
| Dimethicone | 4.80 |
| Butylene glycol | 4.80 |
| Acrylates cross polymer | 0.40 |
| Preservative | q.s |
| Fragrance | q.s |
| Water | To 100 |

### Method

The C12-15 alkyl benzoate and Butylmethoxy dibenzoyl methane are mixed together and heated to 75° C. Mixing is continued until a homogeneous mixture is formed followed by cooling to below 40° C. Ethylhexyl methoxycinnamate, Octocrylene and Methylene bis-benzotriazole tetramethylbutylphenol are added and mixed until homogenenous.

The Oil curd, Almond oil, Dimethicone are added to the above sunscreen solution and mixed until a homogeneous oil curd mixture is formed.

Water, Butylene glycol and Acrylates cross polymer are mixed until homogenenous. The Oil Curd mixture is then added with stirring until uniform. The remaining ingredients are added and mix until a homogenenous formulation is formed.

### EXAMPLE 2

### Anti-Ageing Skin Cream

| | % w/w |
|---|---|
| Oil curd | 10.00 |
| Butylmethoxy dibenzoyl methane | 3.00 |
| Ethylhexyl methoxycinnamate | 7.50 |
| Glycerin | 7.00 |
| C12-15 alkyl benzoate | 5.00 |
| Dimethicone | 2.50 |
| Dimethiconol | 0.50 |
| Cyclopentasiloxane | 0.50 |
| PVP/hexadecane copolymer | 1.00 |
| Acrylates/vinyl isododecanoate copolymer | 0.10 |
| Sodium ascorbyl phosphate | 0.25 |
| Butylene glycol | 0.30 |
| Polyacrylamide emulsion | 1.50 |
| Palmitoyl pentapeptide-3 | 0.00015 |
| Tetrasodium EDTA | 0.05 |
| Potassium hydroxide | 0.03 |
| Preservative | q.s |
| Fragrance | q.s |
| Water | To 100 |

### Method

C12-15 alkyl benzoate and Butylmethoxy dibenzoyl methane are mixed together and heated to 75° C. Mixing is continued until a homogeneous mixture is formed followed by cooling to below 40° C. Ethylhexyl methoxycinnamate is then added and mixed until homogenenous. Dimethicone, PVP/hexadecene copolymer are mixed and then acrylates/vinyl isodecanoate copolymer added and homogenised for 2 minutes. To this mixture is added the oil curd and mixed until homogeneous. Dimethicone/polymer solution is then added.

Glycerin, potassium hydroxide, tetrasodium EDTA and butylene glycol are added to water.

The Oil curd mixture is added to the above described water phase prepared above and mixed for 2 minutes. Cyclopentasiloxane, Dimethiconol and Polyacrylamide Emulsion are added and stirred. Lastly, the remaining ingredients are added and mixed until a homogeneous formulation is produced.

### EXAMPLE 3

### Line Correcting Serum

| | % w/w |
|---|---|
| Oil curd | 10.00 |
| Cyclopentasiloxane | 45.00 |
| Butylene glycol | 5.00 |
| Arabinogalactan | 1.00 |
| Dimethicone crosspolymer | 4.50 |
| Cyclohexasiloxane | 4.20 |
| Sodium ascorbyl phosphate | 1.00 |
| Sodium PCA | 0.40 |
| Retinyl palmitate | 0.15 |
| Medicago sativa | 0.15 |
| Lupinus albus | 0.04 |
| Carbomer | 0.03 |
| Palmitoyl oligopeptide | 0.0003 |
| Palmitoyl tetrapeptide-3 | 0.00015 |
| Preservative | q.s |
| Fragrance | q.s |
| Water | To 100 |

### Method

Carbomer and Arabinogalactan are dispersed in water with homogenisation.

The Oil Curd, Dimethicone Crosspolymer, Cyclopentasiloxane and Cyclohexasiloxane are mixed together until homogeneous and then added to the above described water phase with mixing until homogenenous. Lastly, the remaining ingredients are added and mixed until a uniform formulation is produced.

### EXAMPLE 4

### Moisturising Body Exfoliator

| | % w/w |
|---|---|
| Oil curd | 10.00 |
| Polyethylene | 9.75 |
| Glycerin | 5.00 |
| Glyceryl polymethacrylate | 3.35 |
| Butylene glycol | 3.00 |
| Hydroxyethylcellulose | 1.00 |
| Acrylates/C10-30 alkyl acrylate crosspolymer | 0.40 |
| Hydrogenated vegetable oil beads | 0.25 |
| Triethanolamine | 0.45 |
| Preservative | q.s |
| Fragrance | q.s |
| Water | To 100 |

### Method

Acrylates/C10-30 alkyl acrylate crosspolymer is dispersed in water with homogenisation. Glycerin, Glyceryl Polymethacrylate, Butylene Glycol and Triethanolamine are then added with stirring.

The Oil curd is added to the water phase prepared as described above with stirring. The remaining ingredients are added and mixed until a homogeneous formulation is produced.

### EXAMPLE 5

### Facial Cleanser

| | % w/w |
|---|---|
| Oil curd | 10.00 |
| Paraffinum liquidum | 7.00 |
| Glycerin | 3.00 |
| Dimethicone | 2.00 |
| Sodium Citrate | 0.18 |
| Carbomer | 0.15 |
| Potassium hydroxide | 0.035 |
| Preservative | q.s |
| Fragrance | q.s |
| Water | To 100 |

### Method

Carbomer is dispersed in water with homogenisation. Glycerin, Sodium Citrate and Potassium Hydroxide are added with stirring.

The Oil curd, Paraffinum liquidum and Dimethicone are mixed until homogeneous and then added to the water phase prepared as described above with stirring. Lastly, the remaining ingredients are added and mixed until a homogeneous formulation is produced.

### EXAMPLE 6

### Moisture Lotion

| | % w/w |
|---|---|
| Oil curd | 8.00 |
| Glycerin | 3.00 |
| Dimethicone | 1.50 |
| Carbomer | 0.25 |
| Xanthan Gum | 0.20 |
| Tetrasodium EDTA | 0.02 |
| Potassium hydroxide | 0.01 |
| Preservative | q.s |
| Fragrance | q.s |
| Water | To 100 |

### Method

Carbomer and Xanthan Gum are dispersed in water with homogenisation. Glycerin, Tetrasodium EDTA and Potassium Hydroxide are then added with stirring.

The Oil curd and Dimethicone are mixed together until homogeneous and then added to the water phase prepared as described above with stirring. Lastly, all the remaining ingredients are added and mixed until a homogeneous formulation is produced.

### EXAMPLE 7

### Baby Moisture Cream

| | % w/w |
|---|---|
| Oil curd | 8.00 |
| Glycerin | 2.00 |
| Isopropyl Palmitate | 2.00 |
| Carbomer | 0.10 |
| Tetrasodium EDTA | 0.02 |
| Potassium hydroxide | 0.03 |
| Preservative | q.s |
| Fragrance | q.s |
| Water | To 100 |

### Method

Carbomer is dispersed in water with homogenisation. Glycerin, Tetrasodium EDTA and Potassium Hydroxide are then added with stirring.

The Oil curd and Isopropyl Palmitate are mixed until homogeneous and then added to the water phase prepared as described above with stirring. Lastly, all remaining ingredients are added and mix until a homogeneous formulation is produced.

### EXAMPLE 8

### Baby Lotion

| | % w/w |
|---|---|
| Oil curd | 8.00 |
| Carbomer | 0.32 |
| Tetrasodium EDTA | 0.10 |
| Potassium hydroxide | 0.085 |
| Preservative | q.s |
| Fragrance | q.s |
| Water | To 100 |

### Method

Carbomer is dispersed in water with homogenisation. Tetrasodium EDTA and Potassium Hydroxide is then added to the dispersion with stirring.

The Oil curd is added to the water phase prepared as described above with stirring. Lastly, all the remaining ingredients are added and mixed until a homogeneous formulation is produced.

### EXAMPLE 9

### Anti-Cellulite Balm

| | % w/w |
|---|---|
| Oil curd | 8.00 |
| Glycerin | 5.00 |
| Butylene Glycol | 2.00 |
| Glyceryl Polymethacrylate | 1.50 |
| Sodium Polyacrylate | 0.70 |
| Tetrasodium EDTA | 0.05 |
| Sodium Ascorbyl Phosphate | 0.25 |
| Hesperidine Methyl Chalcone | 0.025 |
| Dipeptide-2 | 0.0005 |
| Palmitoyl Tetrapeptide-3 | 0.00015 |
| Preservative | q.s |
| Fragrance | q.s |
| Water | To 100 |

### Method

Sodium Polyacrylate is dispersed in water with homogenisation. Glycerin, Butylene Glycol, Glyceryl Polymethacrylate and Tetrasodium EDTA are then added with stirring.

The Oil curd is added to the water phase prepared as described above with stirring. Lastly, all the remaining ingredients are added and mixed until a homogeneous formulation is produced.

### EXAMPLE 10

### Foundation

| | % w/w |
|---|---|
| Oil curd | 10.00 |
| Glycerin | 5.00 |
| Butylene Glycol | 2.00 |
| Sodium Polyacrylate | 0.70 |
| Tetrasodium EDTA | 0.05 |
| Sodium Ascorbyl Phosphate | 0.25 |
| Pigments | q.s |
| Preservative | q.s |
| Fragrance | q.s |
| Water | To 100 |

### Method

Sodium Polyacrylate is dispersed in water with homogenisation. Glycerin, Butylene Glycol, and Tetrasodium EDTA is then added with stirring.

The Oil curd is added to the water phase prepared as described above with stirring. Lastly, all the remaining ingredients were added and mixed until a homogeneous formulation is produced.

### EXAMPLE 11

### Hair Conditioner

| | % w/w |
|---|---|
| Oil curd | 5.00 |
| Glycerin | 2.00 |
| Polyquaternium-10 | 1.00 |
| Dimethicone | 0.50 |
| Panthenol | 0.40 |
| Sodium Polyacrylate | 0.70 |
| Tetrasodium EDTA | 0.05 |
| Citric acid | 0.05 |
| Benzophenone-4 | 0.02 |
| Preservative | q.s |
| Fragrance | q.s |
| Water | To 100 |

### Method

Polyquaternium-10 and Sodium Polyacrylate are dispersed in water with homogenisation. Glycerin, Citric acid, Benzophenone-4 and Tetrasodium EDTA are added with stirring.

The Oil curd and Dimethicone are mixed together until homogeneous and then added to the water phase prepared as described above with stirring. Lastly, all the remaining ingredients are added and mixed until a homogeneous formulation is produced.

### EXAMPLE 12

### Anti-Frizz Serum

| | % w/w |
|---|---|
| Oil curd | 10.00 |
| Dimethicone | 10.00 |
| Phenyl Trimethicone | 3.00 |
| Dimethicone Crosspolymer | 0.30 |
| Polyacrylamide Emulsion | 2.75 |
| Tetrasodium EDTA | 0.05 |
| Benzophenone-4 | 0.20 |
| Preservative | q.s |
| Fragrance | q.s |
| Water | To 100 |

### Method

Benzophenone-4 and Tetrasodium EDTA are added to water with stirring.

The Oil curd, Dimethicone Phenyl Trimethicone and Dimethcone Crosspolymer are mixed together until homogeneous. Polyacrylamide Emulsion and the Oil Curd Mixture is added to the water phase prepared as described above with stirring. Lastly, all the remaining ingredients are added and mixed until homogeneous.

### EXAMPLE 13

### Straightening Balm

| | % w/w |
|---|---|
| Oil curd | 8.00 |
| Propylene Glycol | 2.00 |
| VP/VA Copolymer | 1.00 |
| Hydroxyethylcellulose | 0.20 |
| Polyquaternium-10 | 0.10 |
| Tetrasodium EDTA | 0.05 |
| Benzophenone-4 | 0.20 |
| Preservative | q.s |
| Fragrance | q.s |
| Water | To 100 |

### Method

Polyquaternium-10 and Hydroxyethylcellulose are dispersed in water with homogenisation. Propylene glycol, Benzophenone-4, VP/VA Copolymer and Tetrasodium EDTA are added with stirring.

The Oil Curd is added to the water phase prepared as described above with stirring. Lastly, all the remaining ingredients are added and mixed until a homogeneous formulation is produced.

Comparison with Commercially Available Washed Safflower Oil Bodies

Sesame curd, prepared as described above, was incorporated in each of the formulations below and the manufacture and properties of the products were evaluated. The formulations were prepared by conventional methods as described generally herein.

Similar formulations containing hemp curd and sunflower curd were also prepared and the properties of the nine resulting products evaluated.

### Skin Lotion

| Material | % w/w | Function/Nature |
|---|---|---|
| Tetrasodium EDTA | 0.05 | Chelating agent/anionic |
| Methyl Hydroybenzoate | 0.2 | Preservative/non-ionic |
| Xanthan gum | 0.1 | Viscosity |
| | | regulator/anionic |
| Glycerin | 5 | Humectant/non-ionic |
| Capric/caprylic | 2 | Emollient/non-ionic |
| triglyceride | | |
| Sodium polyacrylate | 0.6 | Viscosity |
| | | regulator/anionic |
| Propyl Hydroybenzoate | 0.1 | Preservative/non-ionic |
| Phenoxyethanol | 0.5 | Preservative/non-ionic |
| Aqua | 79.45 | Solvent |
| Oil Curd | 5 | Emulsifier |
| C12-15 alkyl benzoate | 2 | Emollient/non-ionic |
| Dimethicone | 2 | Silicone emollient/non- |
| | | ionic |
| Cetyl alcohol | 2 | Viscosity regulator/non |
| | | ionic |

### Hair Conditioner

| Material | % w/w | Function/Nature |
|---|---|---|
| Cetrimonium chloride | 3 | Conditioning agent/ |
| | | cationic |
| Phenoxyethanol | 0.6 | Preservative/non ionic |
| Aqua | 92 | Solvent |
| Oil Curd | 5 | Emulsifier |
| Cetyl alcohol | 4 | Viscosity regulator/non |
| | | ionic |

### Bodywash

| Material | % w/w | Function/Nature |
|---|---|---|
| Sodium Laureth sulphate | 36 | Surfactant/anionic |
| Cocamidopropyl betaine | 4 | Secondary |
| | | surfactant/amphoteric |
| Tetrasodium EDTA | 0.05 | Chelating agent/anioinc |
| Cocamide DEA | 1.5 | Secondary surfactant/ |
| | | non ionic |
| Sodium chloride | 1.5 | Viscosity regulator/ionic |
| Citric acid | 0.08 | PH adjuster/ionic |
| Aqua | 51.07 | Solvent |
| Sodium Hydroxide | 0.05 | Ph adjuster/ionic |
| Oil Curd | 5 | Emulsifier |
| Potassium Benzoate | 0.15 | Preservative/ionic |
| Phenoxyethanol | 0.6 | Preservative/non ionic |

### Results

The sunflower curd yielded a slightly grey-grainy conditioner, whilst it was found that the other eight products were capable of satisfactory manufacture to form a commercially acceptable smooth creamy lotion for the consumer.

The effectiveness of the bodywash was measured by the foam generation. The test consisted of three expert product users washing the hands in a standardized manner followed by collecting the foam produced for visual assessment of the foam volume and structure. The skin feel of the foam was also assessed. A very good foam production was achieved by all three oil curd bodywash formulations compared to the corresponding formulation omitting the oil curd. However, the stability of the product was less than desired after 24 hours.

The emulsion structures of the sesame curd and hemp curd hair conditioners were found to be satisfactory and were better than the corresponding formulations omitting the oil curd. Each oil curd was found to yield a cream/lotion like viscosity, with the hemp yielding the highest viscosity. Each oil curd was found to yield a product having satisfactory stability properties. In particular, the results with these conditioner formulations was unexpected not only yielding an improved emulsion structure, but also being found to be compatible with cationic material in the formulation. This is particularly important in conditioner formulations where cationic materials are an essential component as cationic materials are highly surface active and are known to disrupt liposomal type structures. Microscopic examination of emulsion structure revealed a more even structure and more liquid crystals with products containing the oil curd compared to products in which the oil curd was absent. Liquid crystals are indicative of improved conditioning performance.

In the skin lotion, each oil curd was found to yield a cream/lotion like viscosity, with the hemp again yielding the highest viscosity. Each oil curd was found to yield a product having satisfactory stability properties.

The nine formulations were compared with an oil body emulsion derived from Safflower seeds commercially available from Lonza under the tradename Natrulon. In this emulsion, the oil bodies recovered from the seed material have been washed thoroughly to remove extrinsic materials, especially extrinsic proteins.

It was found that in the case of the bodywash formulation, the safflower oil body emulsion was not capable of being manufactured as the material was incompatible with the detergent materials in the formulation and separation of the oil curd form the remainder of the formulation occurred once stirring had stopped. Product assessment was impossible.

When incorporated in the conditioner formulation, the safflower oil bodies produced an unsatisfactory low viscosity milk-like fluid. The fluid was found to be unstable after 3 days storage at room temperature. Microscopic examination of the safflower washed oil bodies material revealed an emulsion structure which was very mobile, this also being indicative of instability. This was in line with the expected effect of the cationic conditioning agents on the structure and stability of the oil bodies. The emulsion looked very poor in comparison to the conditioner control formulation which contained no curd.

However, when the safflower washed oil bodies material was incorporated in the skin lotion, it was found to be capable of manufacture to produce a smooth, creamy lotion with acceptable stability.

Table 3 provides a summary of compatibility/stability comparison between commercial washed oil body emulsion and oil curd of the present invention.

TABLE 3

| Base | Oil Curd | Oil Body Emulsion |
|---|---|---|
| Detergent | Compatible | Incompatible |
| | Minimally stable | |
| Conditioner | Compatible | Incompatible |
| | Stable | Unstable |
| Skin Lotion | Compatible | Compatible |
| | Stable | Stable |

1-50. (canceled)

51. A formulation comprising
a) a composition comprising oil curd extracted from oleaginous plant tissue, said oil curd comprising an aqueous dispersion of oil bodies and a stability-enhancing amount of associated extrinsic protein material;
b) a body treatment agent; and optionally
c) a carrier therefor.

52. A formulation according to claim 51 wherein said oil curd is extracted from sunflower, sesame, hemp, pumpkin and oilseed rape seed material.

53. A formulation according to claim 51, wherein the composition comprising oil curd is present in an amount from 0.01% to 20% by weight of the formulation.

54. A formulation according to claim 51 wherein said body treatment agent includes a pharmacologically active ingredient selected from the classes of: analgesics/anti-pyretics, anti-asthmatics, antibiotics, anti-depressants, anti-diabetics, anti-fungal agents, anti-hypertensive agents, anti-inflammatories, anti-neoplastics, anti-anxiety agents, immunosuppresants, anti-migraine agents, sedatives/hypnotics, anti-angina agents, anti-arrythmics, anti-arthritis agents, anti-coagulants, thrombolytic agents, anti-convulsants, anti-Parkinson agents, anti-histamines/anti-pruritics, anti-bacterial agents, anti-viral agents, bronchodilators, steroids and hormones, hypoglycaemic agents, hypolipidemic agents, proteins, anti-ulcer/anti-reflux agents, anti-emetics/anti-nausea agents, local anaesthetics, decongestants, tussives, expectorants and insect repellents.

55. A formulation according to claim 51 including one or more skincare or haircare agents, said agent or agents being present in an amount from 1×10⁻⁶% to 90% by weight of the formulation.

56. A formulation according to claim 55 wherein the formulation is a skincare formulation which is a face cream or lotion, an eye cream, a hand cream or lotion, a body cream or lotion, a foot cream or lotion, a lip balm, a cleanser, a moisturiser, a face mask, a serum, a body cleansing cream, a face/body/foot scrub, a balm, a sunscreen cream or lotion, an after-sun cream or lotion, a fake tan cream or lotion and a nappy cream or lotion.

57. A formulation according to claim 55 wherein the formulation is a hair care formulation which is a conditioner, a balm, a wax, a putty, an emulsion spray, a cleansing product or a serum.

58. A formulation according to claim 55 wherein the formulation is a colour cosmetic formulation which is a lipstick, a lip gloss, a foundation, a mascara, an eye cream including eyeshadow or a blusher.

59. A formulation according to claim 55 comprising one or more skincare agents selected from:
anti-acne actives, vitamins, anti-wrinkle or anti-ageing actives, flavonoids and promoters of collagen and/or elastin production, hydroxy acids, anti-oxidants, anti-inflammatories/soothing agents, anti-microbials, skin-lightening actives, anti-fungals, skin conditioners, organic or inorganic sunscreens, plant extracts and the active principals from these, skin identical lipids such as ceramides, moisturisers, emollients, humectants and materials to promote the repair of the skin barrier, anti-cellulite actives, artificial tanning agents, promoters of melanin synthesis, skin tensors, myorelaxants, skin nutrients, sebum regulators, anti-redness actives and regulators of microbial growth, powders, colourings and pigments; exfoliating materials such as polyethylene beads, loofah, crushed apricot seeds, crushed walnut seeds, bamboo powder, aluminium oxide, silica, oatmeal, rice flour, crushed olive stones and poppy seeds.

60. A formulation according to claim 55, comprising one or more haircare agents selected from:
silicones, vitamins, hair conditioners, shine agents, resins, temporary, semi-permanent and oxidative dyes, sunscreens, antioxidants, proteins, amino acids, organic acids, anti-dandruff agents, lipids, moisturisers, humectants, film formers, reducing agents and oxidising agents.

61. A formulation according to claim 51, which comprises an oil-in-water emulsion.

62. A formulation according to claim 51, which comprises one or lipophilic materials selected from the following classes: natural oils, hydrocarbons, emollient esters, silicones, fatty alcohols and waxes.

63. An oil body-containing composition comprising oil curd extracted from oleaginous plant tissue, said oil curd comprising an aqueous dispersion of oil bodies and associated extrinsic protein material.

64. A composition according to claim 63 wherein said oil curd is extracted from sunflower, sesame, hemp, pumpkin and oilseed rape.

65. A composition according to claim 64 wherein said oil curd is extracted from seed material.

66. A composition according to claim 63 wherein said composition consists essentially of oil curd.

67. A method of preparing a composition comprising oil curd extracted from oleaginous plant tissue, said oil curd comprising an aqueous dispersion of oil bodies and associated extrinsic protein material comprising the steps of:
a) extracting oil bodies from oleaginous plant tissue material and combining with an aqueous medium;
b) removing insoluble material;
c) separating out the oil curd; and
d) storing the oil curd,
the process optionally further comprising the addition of a preserving system.

68. A method according to claim 67 wherein step (a) comprises wet-milling the oleaginous plant seeds in an aqueous medium.

69. A method according to claim 67 wherein the plant tissue comprises seed material.

70. A process to prepare a formulation for administration to a mammal comprising the step of incorporating a composition comprising oil curd extracted from oleaginous plant tissue, said oil curd comprising an aqueous dispersion of oil bodies and a stability-enhancing amount of associated extrinsic protein material in said formulation.

71. A process according to claim 70 wherein said formulation is a cosmetic or dermatological formulation comprising at least one skincare and/or haircare ingredient and a topically acceptable carrier therefor.

72. A process according to claim 70, wherein said formulation comprises a body treatment agent comprising at least one ingredient selected from a cosmetic ingredient and a pharmacologically active ingredient, and a carrier therefor.

73. A process according to claim 70, wherein said composition comprising oil curd is present to emulsify said formulation.

74. A process according to claim 73, wherein said composition comprising oil curd is the sole emulsifier present in the formulation.

75. A process according to claim 70 to prepare a pharmaceutical formulation in the form of a tablet or a capsule.

76. A process according to claim 70, wherein the composition comprising oil curd is present in an amount from 0.01% to 20% by weight of the formulation.

77. A process according to claim 70, comprising the further step of combining said composition comprising oil curd with a cationic material.

78. A process for preparing a formulation for administration to a mammal comprising the steps of
a) extracting oil curd from oleaginous plant tissue, wherein said oil curd comprises an aqueous dispersion of oil bodies and associated extrinsic protein material; and
b) combining said oil curd with a carrier therefor.

79. A process according to claim 78, wherein said oil curd is extracted from oleaginous seeds.

80. A composition according to claim 63, wherein said oil curd is extracted from oleaginous tissue selected from the group consisting of flax, sunflower, sesame, hemp, pumpkin, oilseed rape, camelina, cranberry, safflower, rosehip, blackcurrant, evening primrose, borage, meadowfoam, grapeseed and jojoba.

81. A composition according to claim 63, wherein said oil curd is extracted from sunflower, sesame, hemp, pumpkin and oilseed rape.

82. A composition according to claim 63, wherein said oil curd is extracted from oleaginous seeds.

83. A composition according to claim 63, wherein said oil bodies comprise a lipid core surrounded by a layer comprising phospholipid material and intrinsic protein material and further wherein the amount of extrinsic protein material present in the oil curd exceeds the amount of intrinsic protein material present in the oil curd.

84. A composition according to claim 83, wherein said oil curd comprises a total intrinsic and extrinsic protein content from 1% to 35% by weight of the composition.

85. A composition according to claim 83, wherein said oil curd comprises an extrinsic protein content of from 1% to 35% by weight of the composition.

86. A composition according to claim 83, wherein said lipid is present in an amount less than 50% by weight of the composition.

87. A composition according to claim 83, wherein the ratio of lipid to total protein content in said oil curd is in the range 30:1 to 1:2 parts by weight.

88. A composition according to claim 83, wherein the ratio of lipid to extrinsic protein material is in the range 20:1 to 1:1 parts by weight.

89. A composition according to claim 88, wherein the ratio of lipid to extrinsic protein material is in the range 10:1 to 1:1 parts by weight.

90. A composition according to claim 63, wherein said oil curd comprises carbohydrate material present in the aqueous phase.

91. A composition according to claim 63, wherein said composition comprises a preserving system, preferably in an amount of from 0.001% to 5% by weight of the composition.

92. A composition according to claim 63, wherein said composition comprises from 90% to 100% oil curd by weight of the composition.

93. A composition according to claim 63, wherein said composition comprises at least a portion of exogenous water.

94. A composition according to claim 63, wherein said composition consists essentially of oil curd.

95. A cosmetic treatment method comprising the administration to a mammal in need thereof of a formulation comprising an oil-body-containing composition, said composition comprising oil curd extracted from oleaginous plant tissue wherein said oil curd comprises an aqueous dispersion of oil bodies and a stability-enhancing amount of associated extrinsic protein material.

96. A process according to claim 95, wherein the formulation further comprises at least one pharmacologically active ingredient, and said process is to provide an improved delivery of said at least one pharmacologically active ingredient.

97. A process according to claim 95, to regulate the condition of mammalian skin and/or hair.

98. A process according to claim 95, to improve the appearance of the skin and/or hair.

99. A process according to claim 95, to protect the hair and/or skin from environmental and/or chemical challenge

100. A process according to claim 74, wherein the oil-body containing composition is present to improve the aesthetic acceptability of said formulation.

101. A method of treating a surface of skin and/or hair of a mammal in need thereof comprising the application to said surface of a composition comprising oil bodies, wherein said composition comprises oil curd extracted from oleaginous plant tissue and further wherein said oil curd comprises an aqueous dispersion of oil bodies and a stability-enhancing amount of associated extrinsic protein material.
